(19) United States
(12) Patent Application Publication
(10) Pub. No.: US 2024/0366820 A1
(43) Pub. Date: Nov. 7, 2024

(54) Devices Incorporating Decontamination Means Therein

(71) Applicant: Tesla UV Decontamination Systems Pty Ltd., Wetherill Park (AU)

(72) Inventor: Lou TASIC, Wetherill Park (AU)

(73) Assignee: Tesla UV Decontamination Systems Pty Ltd., Wetherill Park, NS (AU)

(21) Appl. No.: 18/034,581

(22) PCT Filed: Oct. 28, 2021
(86) PCT No.: PCT/AU2021/051254

§ 371 Date: Apr. 28, 2023

(30) Foreign Application Priority Data
Oct. 28, 2020 (AU) 2020903911
Dec. 24, 2020 (AU) 2020904846
Mar. 5, 2021 (AU) 2021900635

(51) Int. Cl. A61L 2/10; A61L 2/24

(52) U.S. Cl. CPC A61L 2/10; A61L 2/24; A61L 2202/122; A61L 2202/14

(57) ABSTRACT

The present invention relates to a system for decontaminating an object. The system comprises a power source, and at least one decontamination area, which includes one or more light sources that emit UV-C radiation to decontaminate the object's surface. In one embodiment, the device comprises the power source; and the at least one decontamination area. In another embodiment, the object comprises the power source; and the at least one decontamination area. In this embodiment, the object further includes a contact area which can become contaminated; and the decontamination area is located within another portion of the object, and is accessible only when the object has undergone a transformation from one use state to another.

### PRIORITY DETAILS

The present application claims priority from AU 2020903911, filed in Australia on 28 Oct. 2020, AU 2020904846, filed in Australia on 24 Dec. 2020, and AU 2021900635, filed in Australia on 5 Mar. 2021, the contents of which are incorporated herein in their entirety by cross-reference.

### TECHNICAL FIELD

The present invention relates generally to the field of devices used to minimise or prevent the spread of infectious agents, such as bacteria and viruses to people by them touching surfaces.

In particular, the present invention relates to a device that minimises or prevents the spread of infectious agents and reduces the contamination of EFTPOS (Electronic Funds Transfer at Point of Sale) and ATM (Automated Teller Machine) terminals. The present invention also relates to devices for use in possibly contaminating environments, which incorporate a decontamination means in order to decontaminate the portion(s) of the devices that are most likely to become contaminated when using the device. The integrated decontamination means minimises or prevents the spread of infectious agents and reduces the possibility of a user being contaminated by using the device.

### BACKGROUND

Whilst spreading of infectious agents such as viruses and bacteria via surface contamination is always a factor of disease spread, it is of particular concern during global disease pandemics, such as the current SARS-CoV-2 pandemic.

Many potentially infectious bacteria, viruses, yeasts and moulds can survive on surfaces for considerable amounts of time. For example, in coronavirus studies, the most resilient viruses took up residence on non-porous plastic and steel. Infectious particles have been detected on both types of surfaces commonly for up to 72 hours and, in some cases on steel for up to 28 days. Of particular concern when it comes to surface transmission, non-porous surfaces that we touch often but rarely clean remain a concern. This is particularly prevalent when it comes to non-porous surfaces that are touched by many people in quick succession, which greatly increases the possibility of infection occurring.

Devices that fall into this category of higher risk infection points include EFTPOS and ATM terminals, lift buttons and others because they are manufactured from non-porous material like plastic and metal, are high touch point devices by many people and are used frequently, and in quick succession by multiple people.

Traditionally, ways to minimise the transmission of infectious agents have been frequent cleaning of the EFTPOS and ATM terminals—and particularly the keypads, which are frequently touched by user's fingers and hands. However, frequent cleaning of these terminals with liquid chemicals can cause unintentional damage to the machines, or at least lead to shorter than normal lifespan of the devices because they were not designed with such frequent cleaning in mind.

Alternatively, it has been left up to individual users of these terminals to take responsibility for their own hygiene, and to sanitize their hands both before and after using such devices, which is done by thorough washing of their hands (which is not always possible where the terminals are located) or by alcohol based or other suitable type hand sanitizer lotions, sprays or wipes. However, sometimes a user may not have access to a suitable hand sanitizer, and in some cases not all users take good responsibility for hand cleanliness, and this can lead to an increased risk of infectious transmission. Although alcohol-based hand sanitizers can inactivate many types of microbes very effectively when used correctly 1-15, people may not use a large enough volume of the sanitizers or may wipe it off before it has dried, accordingly, there is a possibility that the use of hand sanitizer does not eliminate all microbes, bacteria and viruses.

Another consideration of the present invention arises out of concerns in using devices such as pens and electronic device stylus, which can be generally categorized as high touch objections, particularly if used in public areas. Perversely, during viral disease pandemics it is not uncommon for venues to insist upon patrons/customers ‘signing in’ to record their details for possible later use in contract tracing by health officials if there are positive infections recorded, and when done using conventional pens, this may be a source of infection. In other cases, there are devices that require cleaning and decontamination after each use, such as sexual stimulation devices. The present invention can be utilised in a more efficient and effective cleaning regime for such devices.

Traditionally, it has been left up to individual users of these devices to take responsibility for their own hygiene, and to sanitize their hands both before and after using such devices, which is done by thorough washing of their hands (which is not always possible where the devices are used) or by alcohol based or other suitable type hand sanitizer lotions, sprays or wipes. However, sometimes a user may not have access to a suitable hand sanitizer, and in some cases not all users take good responsibility for hand cleanliness, and this can lead to an increased risk of infectious transmission. Although alcohol-based hand sanitizers can inactivate many types of microbes very effectively when used correctly 1-15, people may not use a large enough volume of the sanitizers or may wipe it off before it has dried, accordingly, there is a possibility that the use of hand sanitizer does not eliminate all microbes, bacteria and viruses.

Accordingly, there exists a need to provide an alternate solution to this problem of surface contamination and prevent the spread of infectious agents between people using high touch point devices, such as EFTPOS and ATM terminals, lift buttons, etc., or at least provide a suitable alternative thereto, and to provide a more efficient and effective way of decontaminating devices that are used in possibly contaminating environments

### DISCLOSURE OF THE INVENTION

In a first aspect, the present invention relates to a system for decontaminating an object, the system comprising a power source, and at least one decontamination area; and characterized in that the at least one decontamination area includes one or more light sources that emit UV-C radiation.

Preferably, in accordance with a first embodiment of the invention, the system includes a device for decontaminating a surface of the object, wherein the device incorporates the power source and the at least one decontamination area, and said device decontaminates the surface of the object when the object is located in the decontamination area and the one or more light sources is activated.

Preferably, in accordance with a second embodiment of the invention, the object can be provided for use in a possibly contaminated environment, and the object is configured to incorporate the power source and the at least one decontamination area, wherein the object further comprises a contact area, which can become contaminated when the object is used within the possibly contaminated environment; and wherein the at least one decontamination area is located within a portion of the object other than the contact area, and is accessible only when the object has undergone a transformation from one use state to another, and the decontamination area includes a void into which the contact area of the object is placed, and further include the one or more light sources which decontaminate the contact area of the object when activated.

### DESCRIPTION OF FIGURES

Embodiments of the present invention will now be described in relation to figures, wherein

FIG. 1 depicts a device for decontaminating a surface of an object in accordance with a first preferred embodiment of the present invention, where the object is a user's finger;

FIG. 2 depicts the device shown in FIG. 1, showing how a user's finger enters the decontamination area;

FIG. 3a depicts a device for decontaminating a surface of an object in accordance with a second preferred embodiment of the present invention, which has both a finger decontamination area and a bank card decontamination area, and where the object is a user's finger or a bank card;

FIG. 3b depicts an alternate embodiment of a device for decontaminating a surface of an object in accordance with a second preferred embodiment of the present invention, which has both a finger decontamination area and a bank card decontamination area, and where the object is a user's finger or a bank card;

FIG. 4a depicts a device for decontaminating a surface of an object in accordance with a third preferred embodiment of the present invention, where the object is an EFTPOS terminal;

FIG. 4b depicts an alternate embodiment of a device for decontaminating a surface of an object in accordance with a third preferred embodiment of the present invention, where the object is an EFTPOS terminal;

FIG. 4c depicts an alternate embodiment of a device for decontaminating a surface of an object in accordance with a third preferred embodiment of the present invention, where the object is an EFTPOS terminal, although it is not shown;

FIG. 4d depicts an alternate embodiment of a device for decontaminating a surface of an object, shown with power source, in accordance with a third preferred embodiment of the present invention, where the object is the playing surface of a gaming machine, although it is not shown;

FIG. 5 depicts an alternate embodiment of a device for decontaminating a surface of an object in accordance with a fourth preferred embodiment of the present invention, similar to the embodiment shown in FIG. 4a and FIG. 4b, where the object is an EFTPOS terminal, although it is not shown;

FIG. 6 depicts an alternate embodiment of a device for decontaminating a surface of an object in accordance with a fourth preferred embodiment of the present invention, similar to the embodiment shown in FIG. 4a, FIG. 4b and FIG. 5, where the object is an EFTPOS terminal, although it is not shown;

FIG. 7 depicts a device for decontaminating a surface of an object in accordance with another preferred embodiment of the present invention, where the object is a user's finger, although it is not shown;

FIG. 8a depicts a device for decontaminating a surface of an object in accordance with a fifth preferred embodiment of the present invention, where the object is a user's hand, although it is not shown;

FIG. 8b depicts an alternate embodiment of a device for decontamination a surface of an object in accordance with a fifth preferred embodiment of the present invention, where the object is a user's hand, although it is not shown;

FIG. 8c depicts the alternate embodiment of a device for decontamination a surface of an object in accordance with a fifth preferred embodiment of the present invention shown on a stand, and where the object is a user's hand, although it is not shown;

FIG. 9a depicts a decontaminating a surface of an object in accordance with a sixth preferred embodiment of the present invention, similar to the embodiment shown in FIG. 1, where the object is a user's finger, although it is not shown;

FIG. 9b depicts an alternate embodiment of a decontaminating a surface of an object in accordance with a sixth preferred embodiment of the present invention, similar to the embodiment shown in FIG. 1 and FIG. 9a, where the object is a user's finger, although it is not shown;

FIG. 9c depicts an alternate embodiment of a decontaminating a surface of an object in accordance with a sixth preferred embodiment of the present invention, similar to the embodiment shown in FIG. 1, FIG. 9a and FIG. 9b, where the object is a user's finger, although it is not shown;

FIG. 10a depicts a decontaminating a surface of an object in accordance with a seventh preferred embodiment of the present invention, where the object is a shopping trolley;

FIG. 10b shows the seventh preferred embodiment of the present invention of FIG. 10a;

FIG. 10c shows the seventh preferred embodiment of the present invention of FIGS. 10a and 10b, and specifically shows the protective shield thereof;

FIG. 11a depicts a device for decontaminating a surface of an object in accordance with an eighth preferred embodiment of the present invention, where the object is a display monitor, although it is not shown;

FIG. 11b shows the eighth preferred embodiment of the present invention of FIG. 11a, showing the engagement with the display monitor;

FIG. 11c shows the eighth preferred embodiment of the present invention of FIG. 11a and FIG. 11b, showing the operation of the decontamination device with the display monitor;

FIG. 11d depicts an alternate embodiment of a device for decontaminating a surface of an object in accordance with an eighth preferred embodiment of the present invention, where the object is a display monitor, although it is not shown;

FIG. 11e depicts the embodiment of FIG. 11d, and specifically shows the eighth preferred embodiment of the present invention of FIG. 11d, where the component of the device used to move the decontamination portion of the device in the form of a finger rest.

FIG. 12 depicts a device for decontaminating a surface of an object in accordance with an eleventh embodiment of the present invention, where the object is in the form of a pen, and the device is part of the pen;

FIG. 13 depicts the device and object shown in FIG. 21, showing that the decontamination area is located within the pen lid and only visible when the lid is removed from the pen barrel;

FIG. 14 depicts the device and object shown in FIG. 21, showing that the pen barrel can be inserted into the decontamination area located within the pen lid;

FIG. 15a depicts a cross-sectional view of the device and object shown in FIG. 21, showing the writing tip end of the pen inserted into the decontamination area of the pen lid;

FIG. 15b depicts a close up cross-sectional view of the pen lid, showing the details of the decontamination area, consisting of the chamber and the UV-C light source;

FIG. 16 depicts a device for decontaminating a surface of an object in accordance with a twelfth preferred embodiment of the present invention, where the object is in the form of a pen with integrated stylus, and the device is part of the pen with integrated stylus;

FIG. 17 depicts the device and object of FIG. 25, showing that the decontamination area is located at the end of the pen barrel adjacent the stylus, but only visible when the stylus is removed from the pen barrel;

FIG. 18 depicts the device and object of FIG. 25, showing that the stylus can be inserted into the decontamination area located within the pen barrel;

FIG. 19 depicts a cross-sectional view of the device and object shown in FIG. 25, showing the stylus inserted into the decontamination area located in the pen barrel adjacent the location of the stylus;

FIG. 20 depicts a device for decontaminating a surface of an object in accordance with a thirteenth preferred embodiment of the present invention, where the object is in the form of a sexual stimulation device, and the device is part of the sexual stimulation device; and

FIG. 21 depicts a cross-sectional view of the device and object shown in FIG. 29, showing the removable vibrating tip inserted into the decontamination area located in the body of the device adjacent the location of the removable tip.

### DETAILED DESCRIPTION OF PREFERRED EMBODIMENTS

The present invention relates to a system for decontaminating an object. The system can include various devices or objects that are used in possibly contaminating environments or that are responsive to contamination, and which incorporate a decontamination means in order to decontaminate the object.

In some embodiments of the invention, the device is separate to the object and in other embodiments of the invention, the device and the object are integrated with each other. In these embodiments, the portion(s) of the devices that are most likely to become contaminated when using the device in the possibly contaminating environment can then be decontaminated by transforming the device from one use state to another, such that an integrated decontamination area within the device becomes accessible and can then decontaminate another portion of the device.

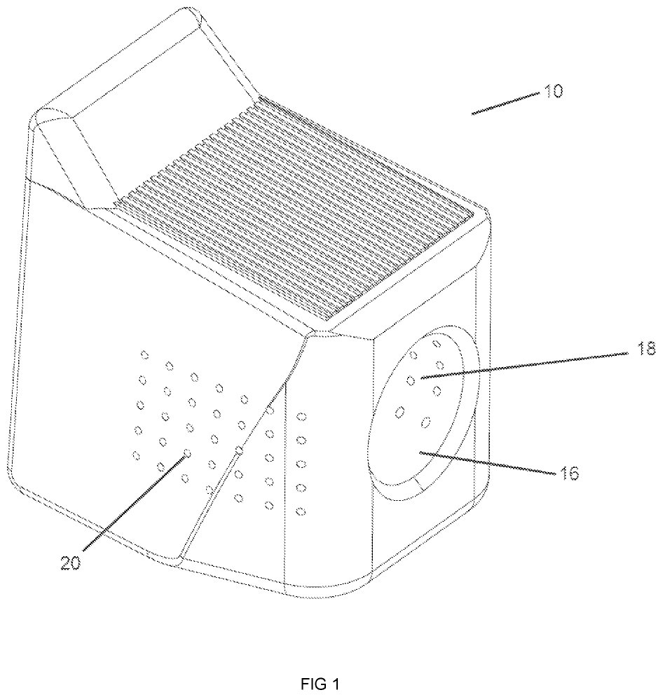

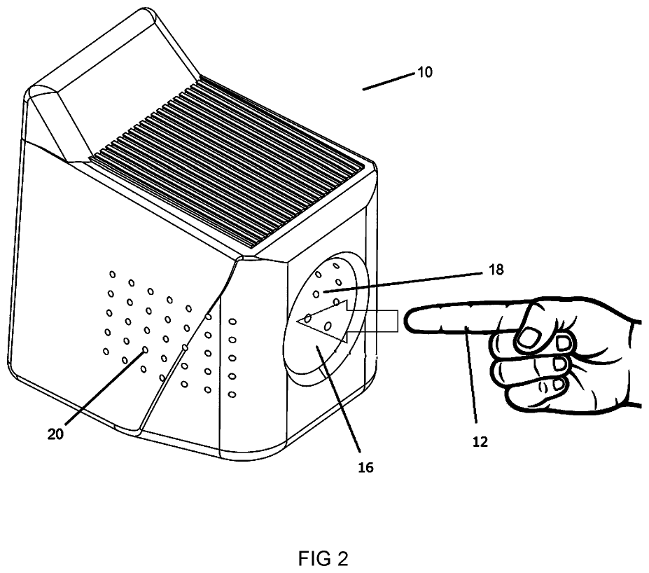

As best shown in FIGS. 1 and 2, a first embodiment of the present invention relates to a device 10 for decontaminating a surface of an object 12, which in this embodiment is a user's finger 12. The device 10 comprises a power source (which is not shown in the drawings), and at least one decontamination area 16, which includes a void into which the surface of the object 12 can be placed or inserted. This is best shown in FIG. 2, where the arrow depicts the direction of the user's finger 12 as it is inserted into the void of the decontamination area 16. The decontamination area 16 further includes one or more light sources 18 that emit UV-C radiation, which decontaminates the surface of the object 12 when activated. The UV-C light source is preferably an array of UV-C emitting LEDs (light emitting diodes).

The use of UV-C radiation to inactivate microorganisms so they are no longer capable or replicating and causing adverse health effects is well known. UV-C radiation has been used for decades to disinfect surfaces and sanitise water and air for example. Ultra-Violet light is invisible to the human eye and is divided into UV-A, UV-B and UV-C. UV-C radiation is found within 100-280 nm range, and its germicidal action is maximized at 254 nm with reductions on either side of this wavelength.

Ultraviolet (UV) is that part of electromagnetic light bounded by the lower wavelength extreme of the visible spectrum and the X-ray radiation band. The spectral range of UV light is, by definition between 100 and 400 nm (1 nm=10−9 m) and is invisible to human eyes. Using the CIE classification the UV spectrum is subdivided into three bands:
UV-A (long-wave) from 315 to 400 nm
UV-B (medium-wave) from 280 to 315 nm.
UV-C (short-wave) from 100 to 280 nm

UV-C radiation works using a photolytic effect whereby the radiation destroys or inactivates the micro-organism so that it can no longer multiply. For DNA it does this by causing adjacent thymine bases to form a chemical bond thus creating a dimmer and if sufficient of these are created, DNA cannot replicate.

The UV light emitted by a source is expressed in watts (W) and the irradiation density is expressed in watts per square meter (W/m2). For germicidal action dose is important. The dose is the irradiation density multiplied by the time (t) in seconds and expressed in joules per square meter (J/m2)·(1 joule is 1 W·second).

Micro-organisms effective resistance to UV light varies considerably. Moreover, the environment of the particular micro-organism greatly influences the radiation dose needed for its destruction.

The survival of micro-organisms when exposed to UV light is given by the approximation:

Nₜ/N₀=exp. (−kE_{eff}t)
Nₜ is the number of germs at time t
N₀ is the number of germs before exposure
k is a rate constant depending on the species
E_{eff} is the effective irradiance in W/m2
   The product E_{eff} is called the effective dose
H_{eff} and is expressed in W·s/m2 of J/m2
   Some k value indications are given in Table 1, where they can be seen to vary from 0.2 m2/J viruses and bacteria, to 2.10-3 for mold spores and 8.10-4 for algae.

TABLE 1
Doses for 10% survival under 254 nm
radiation (J/m2) and rate constant k (m2/J)
UV dose to obtain 90% killing rate

| | Dose | k |
|---|---|---|
| Bacteria | | |
| Bacillus anthracis | 45.2 | 0.051 |
| B. megatherium sp. (spores) | 27.3 | 0.084 |
| B. megatherium sp. (veg.) | 13.0 | 0.178 |
| B. parathyphosus | 32.0 | 0.072 |
| B. suptilis | 71.0 | 0.032 |
| B. suptilis spores | 120.0 | 0.019 |
| Campylobacter jejuni | 11.0 | 0.209 |
| Clostridium tetani | 120.0 | 0.019 |
| Corynebacterium diphteriae | 33.7 | 0.069 |
| Dysentery bacilli | 22.0 | 0.105 |
| Eberthella typhosa | 21.4 | 0.108 |
| Escherichia coli | 30.0 | 0.077 |
| Klebsiella terrifani | 26.0 | 0.089 |
| Legionella pneumophila | 9.0 | 0.256 |
| Micrococcus candidus | 60.5 | 0.038 |
| Micrococcus sphaeroides | 100.0 | 0.023 |
| Mycobacterium tuberculosis | 60.0 | 0.038 |
| Neisseria catarrhalis | 44.0 | 0.053 |
| Phytomonas tumefaciens | 44.0 | 0.053 |
| Pseudomonas aeruginosa | 55.0 | 0.042 |
| Pseudomonas fluorescens | 35.0 | 0.065 |
| Proteus vulgaris | 26.4 | 0.086 |
| Salmonella enteritidis | 40.0 | 0.058 |
| Salmonella paratyphi | 32.0 | 0.072 |
| Salmonella typhimurium | 80.0 | 0.029 |
| Sarcina lutea | 197.0 | 0.012 |
| Seratia marcescens | 24.2 | 0.095 |
| Shigella paradysenteriae | 16.3 | 0.141 |
| Shigella sonnei | 30.0 | 0.077 |
| Spirillum rubrum | 44.0 | 0.053 |
| Staphylococcus albus | 18.4 | 0.126 |
| Staphylococcus aureus | 26.0 | 0.086 |
| Streptococcus faecalis | 44.0 | 0.052 |
| Streptococcus hemoluticus | 21.6 | 0.106 |
| Streptococcus lactus | 61.5 | 0.037 |
| Streptococcus viridans | 20.0 | 0.115 |
| Sentertidis | 40.0 | 0.057 |
| Vibrio chlolerae (V. comma) | 35.0 | 0.066 |
| Yersinia enterocolitica | 11.0 | 0.209 |
| Yeasts | | |
| Bakers' yeast | 39 | 0.060 |
| Brewers' yeast | 33 | 0.070 |
| Common yeast cake | 60 | 0.038 |
| Saccharomyces cerevisiae | 60 | 0.038 |
| Saccharomyces ellipsoideus | 60 | 0.038 |
| Saccharomyces sp. | 80 | 0.029 |
| Mould spores | | |
| Aspergillus flavus | 600 | 0.003 |
| Aspergillus glaucus | 440 | 0.004 |
| Aspergillus niger | 1320 | 0.0014 |
| Mucor racemosus A | 170 | 0.013 |
| Mucor racemosus B | 170 | 0.013 |
| Oospora lactis | 50 | 0.046 |
| Penicillium digtatum | 440 | 0.004 |
| Penicillium expansum | 130 | 0.018 |
| Penicillium roqueforti | 130 | 0.018 |
| Rhizopus nigricans | 1110 | 0.002 |
| Virus | | |
| Hepatitis A | 73 | 0.032 |
| Influenza virus | 36 | 0.064 |
| MS-2 Coliphase | 186 | 0.012 |
| Polio virus | 58 | 0.040 |
| Rotavirus | 81 | 0.028 |
| Protozoa | | |
| Cryptosporidium parvum | 25 | 0.092 |
| Giardia lamblia | 11 | 0.209 |
| Algae | | |
| Blue Green | 3000 | 0.0008 |
| Chlorella vulgaris | 120 | 0.019 |

Whilst there a strong germicidal effect is provided by the light radiation in the short-wave UV-C band, there are also health and safety risks to consider although they are minor. For example, erythema (reddening of the skin) and conjunctivitis (inflammation of the mucous membranes of the eye) can be caused by this form of light radiation. Because of this, when germicidal UV-Light lamps are used, it is important to design systems to exclude UV-C exposure and so avoid these effects. The designs of the present invention have taken these health and safety concerns into consideration when the devices of the present invention were designed.

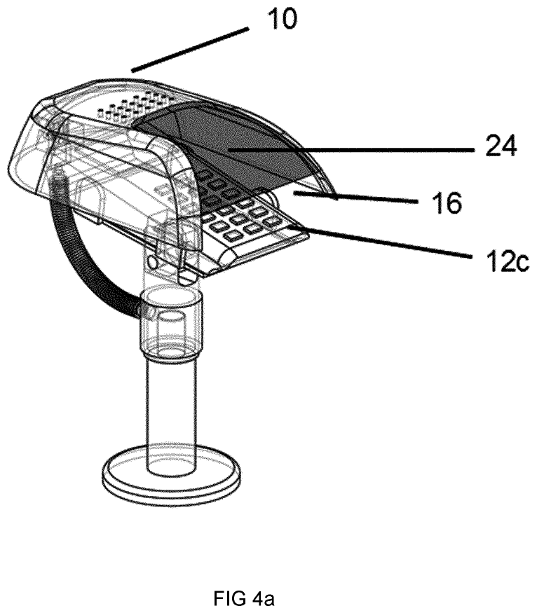

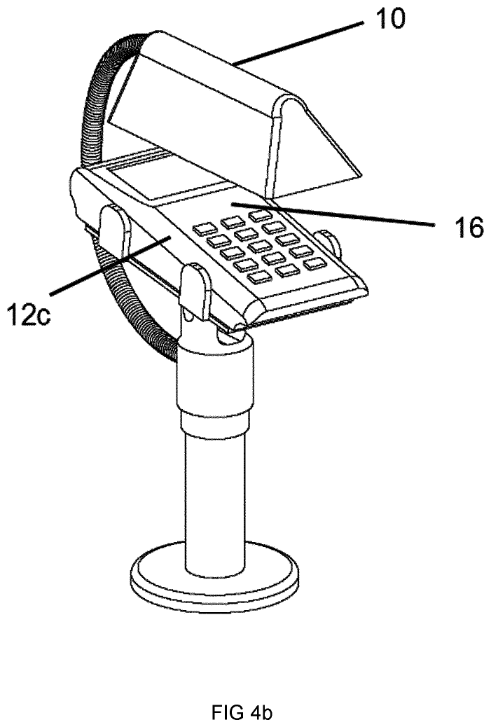

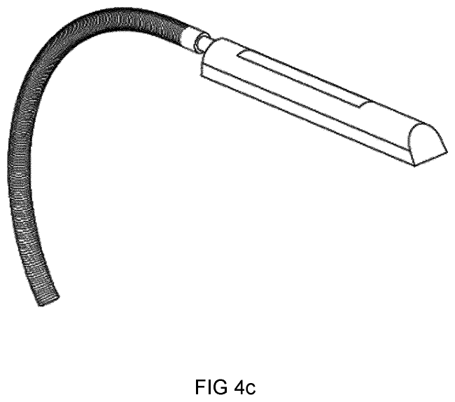

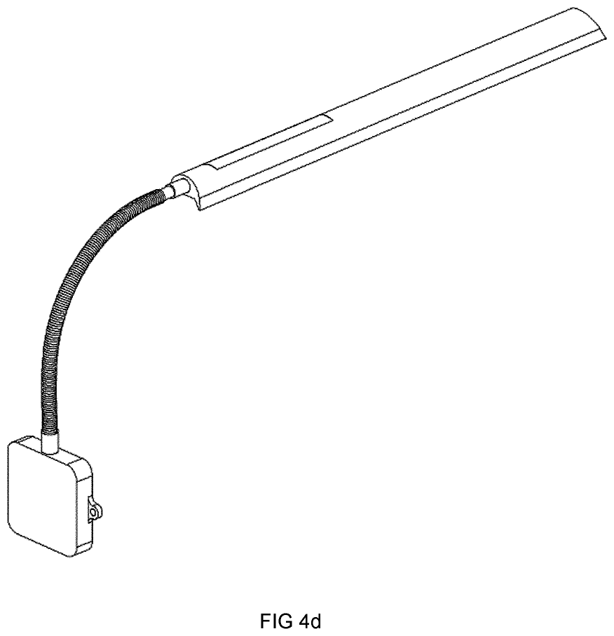

For example, as is particularly well shown in the embodiments of the present invention shown in FIGS. 4a, 4b, 4c, 4d, 5, 6, 9a and 10c, the decontamination area 16 of the device 10 can include a shield 24 at least partially obstructing visual access into the void in order to protect the user's eyes from the UV-C radiation, which can be damaging. The UV-C radiation cannot penetrate shield 24, and so provides a shield to the user from being exposed to UV-C radiation. However, the shield itself may be at least partially transparent so that the user can see into the decontamination area, as the frequency of UVC light waves are sufficiently small that they do not readily pass through material, so even a substantially transparent shield material will be useful for this purpose.

In addition, the device 10 for decontaminating a surface of an object can in some instances further comprise a movement sensor that is designed to detect if/when the device has been moved. When the sensor makes such a detection, it is connected with the UV-C light source, and will automatically deactivate the UV-C radiation to ensure that there is no accidental and unintended exposure of UV-C radiation on a person.

Whilst it is not shown in the drawings, the decontamination area 16 can further include an automated sensor to detect when the object is introduced therein, When the object is detected, the sensor will automatically activate the one or more the light sources 18.

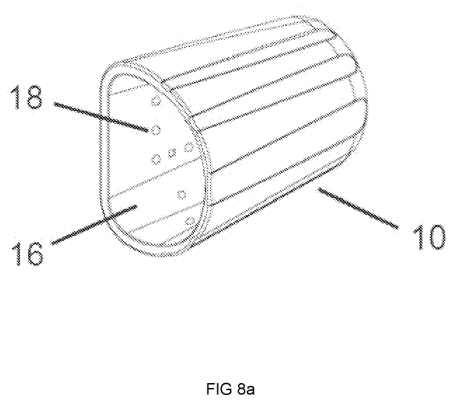

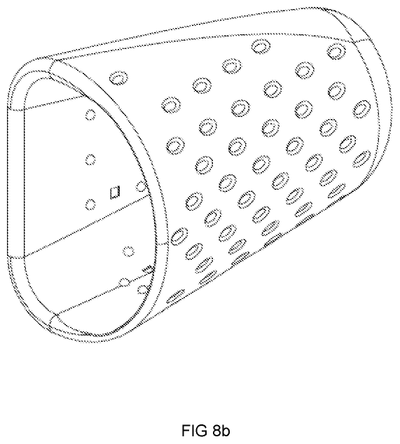

This is particularly useful for the embodiment of the present invention shown in FIGS. 1-3, FIG. 7 and FIGS. 9a,9b and 9c, where the user inserts their finger into the decontamination area 16, or in FIGS. 8a, 8b and 8c, where the user inserts their hand into the decontamination area, and can activate the UV-C light without having to touch the device 10. To provide a greater sense of user satisfaction, the device 10 further includes a second light source (that is not shown in the drawings) that emits visible light when activated. This light source is designed to illuminate at the same time the UV-C radiation is being emitted by the UV-C light source, and thus the visible light indicates to a user when the UV-C light source is activated. This light is preferably bright violet or blue, and most preferably is the visible spectrum UV-A or UV-B light, which user's associate with sterilizing and decontamination.

This second visible light source is located within the decontamination area 16 such that the void, and the object positioned therein is illuminated by the visible light simultaneously with the UV-C radiation emitted from the UV-C light source.

As shown in Table 1 above, the different intensities and time period for irradiation is shown for different infectious agents. Given where the devices of the present invention are to be deployed, and the purpose of the devices, as detailed above, where the object is a user's finger, it is intended that the UV-C light source is activated automatically when the user insert's their finger within the void of the decontamination area for approx. 10 seconds. However, where the object is an EFTPOS terminal, it will be decontaminated for a set period of time as per the timer setting frequency, which is preferably set at a time period of two (2) minutes. However, for safety, if movement is detected during that time period, the UV-C emitting lights will be automatically deactivated, and after a set period that can be adjusted to suit individual operator needs, will subsequently be re-activated and complete the decontamination. At the conclusion of the decontamination time period, the UV-C emitting lights will automatically be deactivated.

The device 10 for decontaminating a surface of an object can further comprise a digital display (that is not shown in the drawings) that conveys information about the duration of the period of activation of the UV-C light source to the user in real time. The digital display may, for example, includes a countdown timer showing the duration of the period of activation of the at least one UV-C light source 18.

Embodiments of the device 10, 20 of the present invention, which are designed primarily for use in decontaminating extremities of a user, such as fingers, hands and the like are shown in FIGS. 1 to 9c. In these various embodiments, a user can insert parts of their body (like a finger or a hand) into the decontamination area 16 of the device 10, 20.

As is shown in FIGS. 1-2, in one preferred embodiment of the present invention, the device 10 is directed to a scenario in which the object 12 is a users' finger.

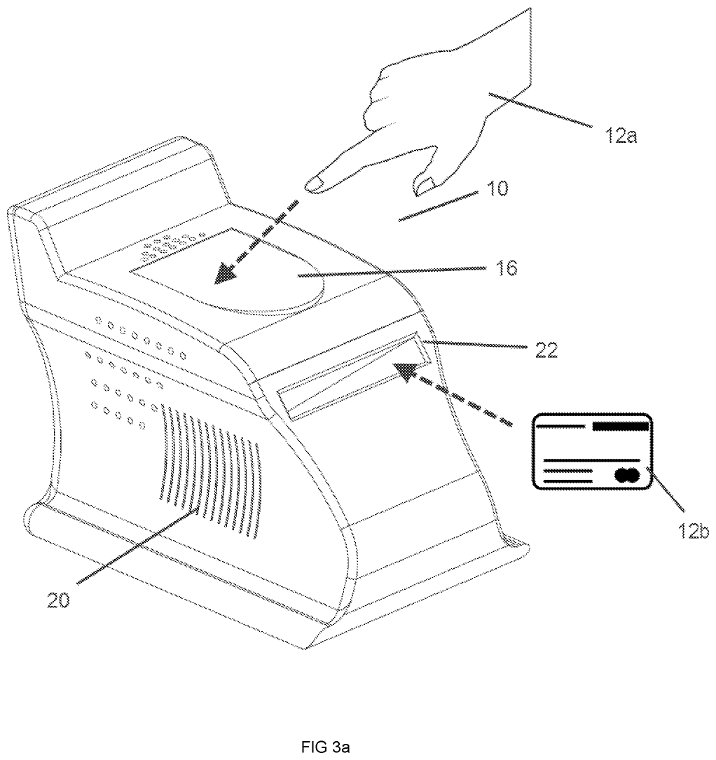

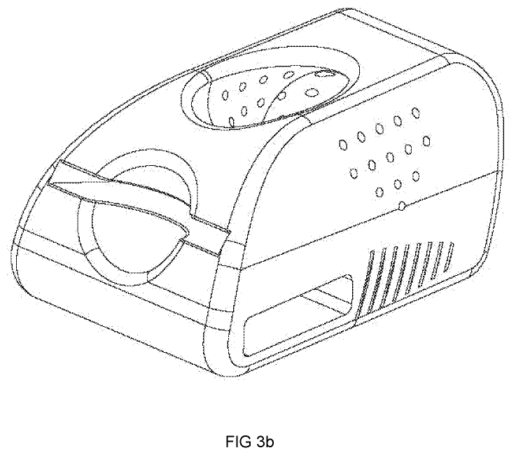

A slightly different embodiment is shown in FIG. 3, in which the device 10 includes two decontamination areas, the first 16 being for the user to insert their finger 12a, and the second 22 being for a user to insert their payment card 12b. It is also envisaged that the user may choose to insert their driver's license or any other card to decontamination if desired.

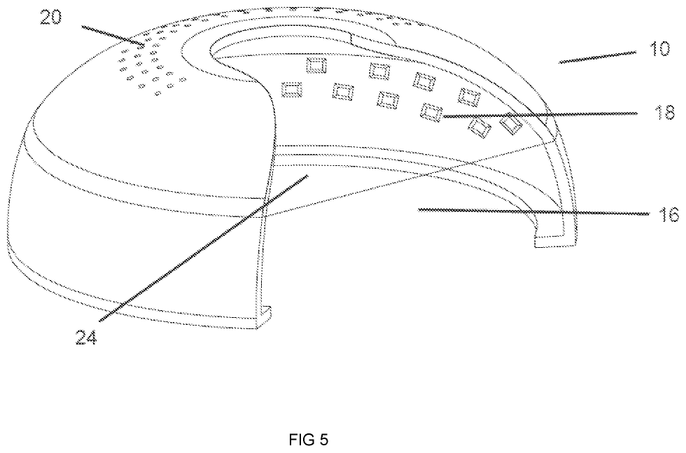

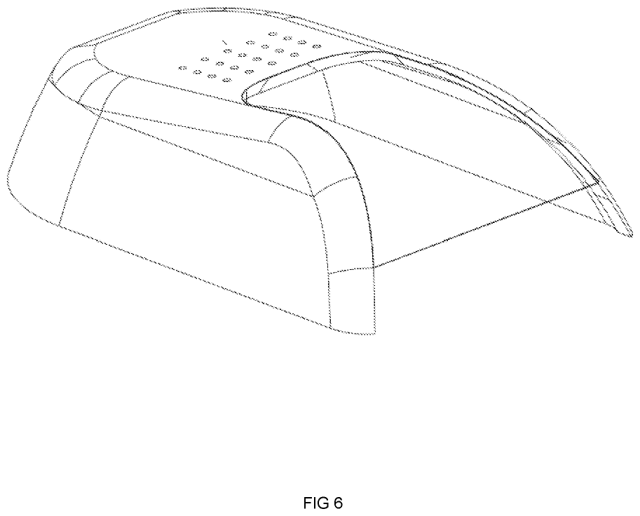

In third, fourth and fifth embodiments of the present invention shown in FIGS. 4a, 4b, 4c, 4d, 5 and 6, the device 10 and the device 20 are designed for use with an object 12 in the form of an EFTPOS terminal 12c. The embodiments shown in FIGS. 4a and 4b are designed for best use with an EFTPOS terminal on a stand (as shown), whereas the embodiments shown in FIGS. 5 and 6 are designed for best used with an EFTPOS terminal that does not have a stand, in which case both the EFTPOS terminal and the device 10, 20 will simply sit on a bench, table or point of sale area or display. These particular embodiments of the devices of the present invention are also particularly well suited for other uses, and can also be used to decontaminate telephones (especially mobile or cellular telephones), game machine controls, etc., which can be placed in one of the decontamination areas of the device.

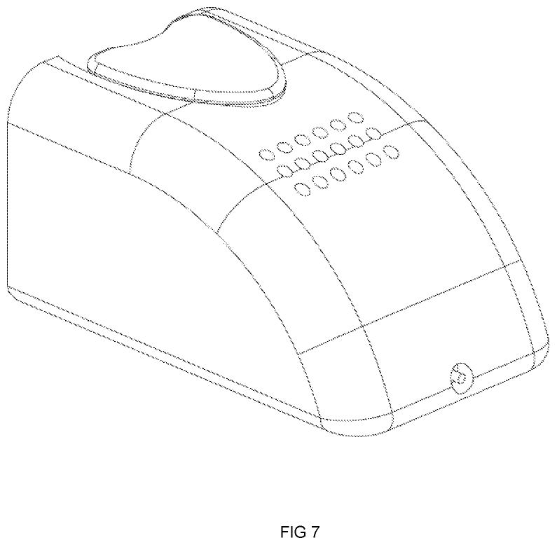

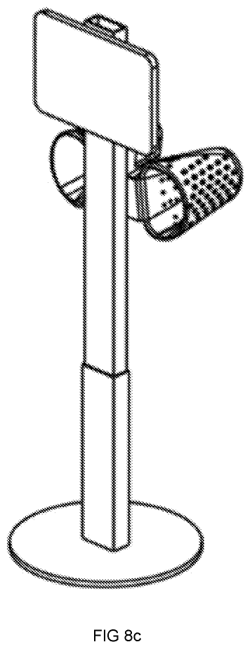

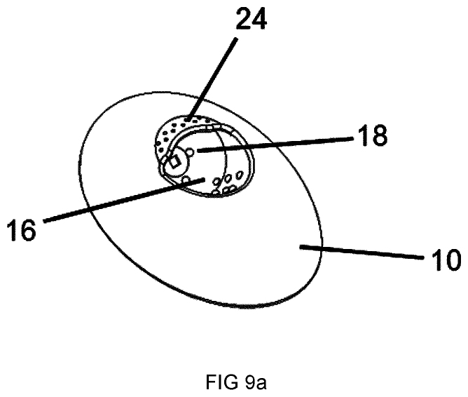

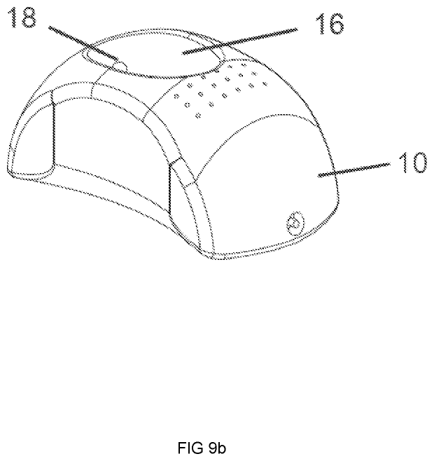

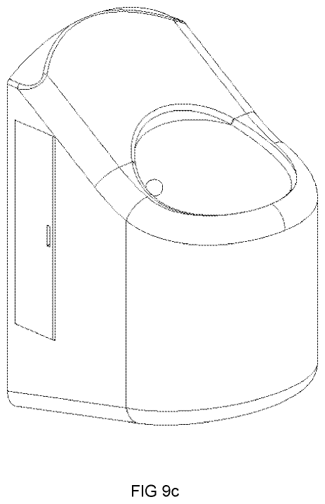

Another embodiment of the device 10 of the present invention is shown in FIG. 7, which is similar to that shown in FIGS. 1-2 in that the device is designed for an object 12 being a users' finger. Further similar embodiments are shown in FIGS. 9a, 9b and 9c, and are designed for decontaminating a users' finger.

A further embodiment of the device 10 of the present invention is shown in FIGS. 8a, 8b and 8c, in which the object 12 is a users' hand.

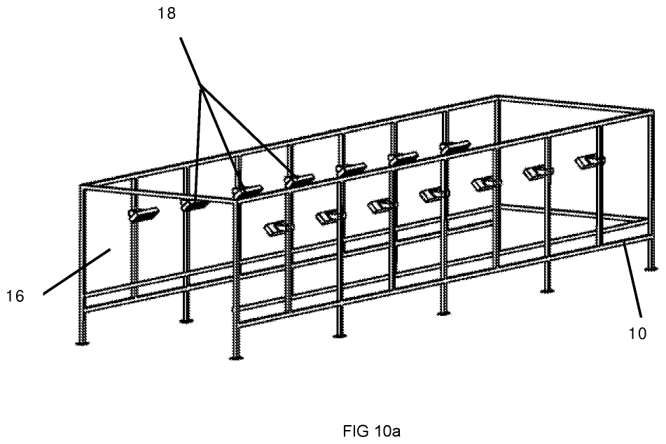

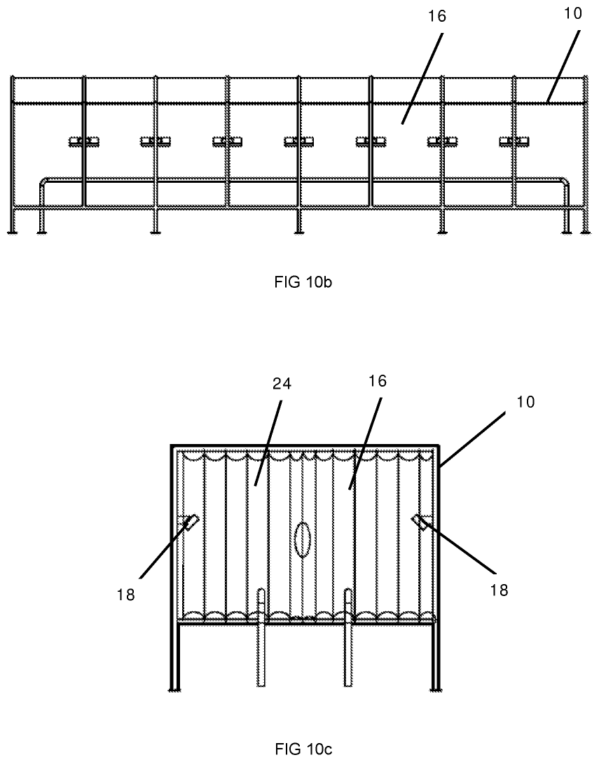

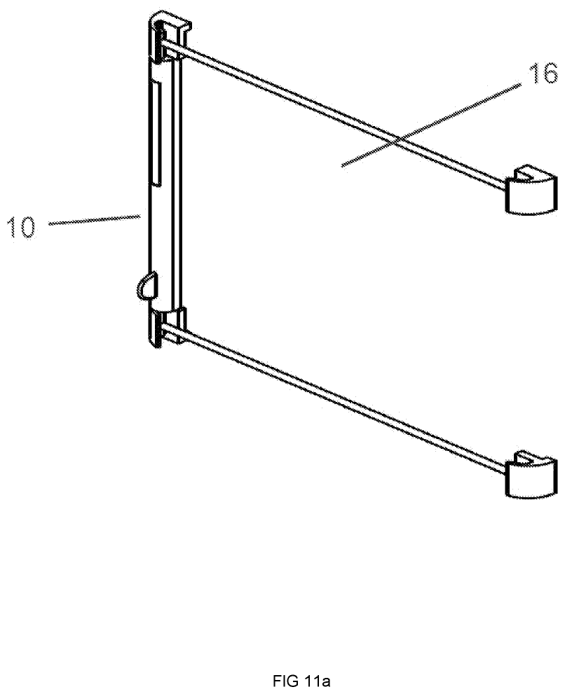

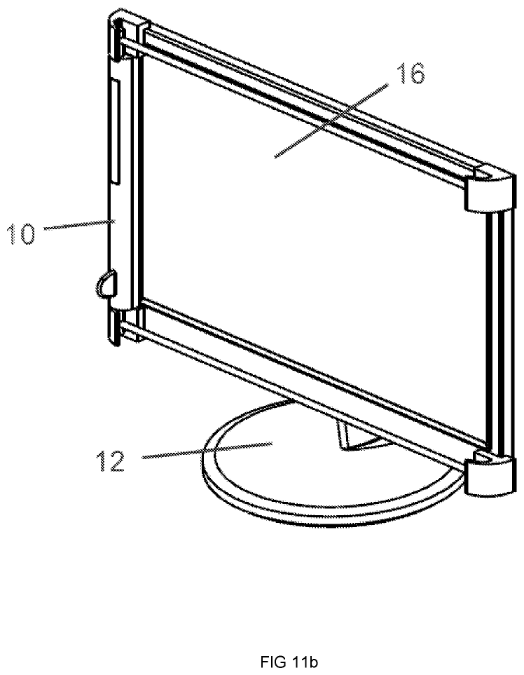

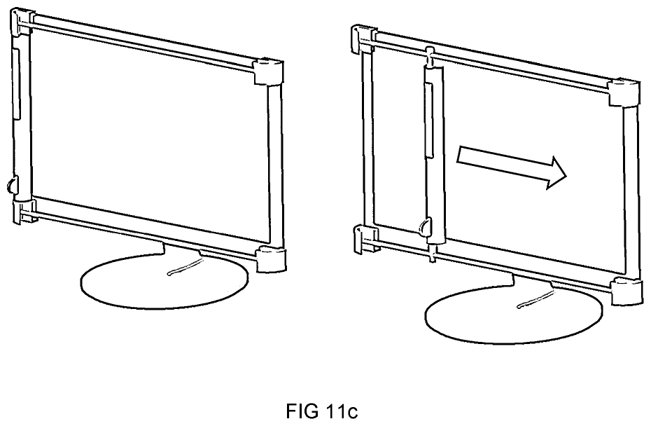

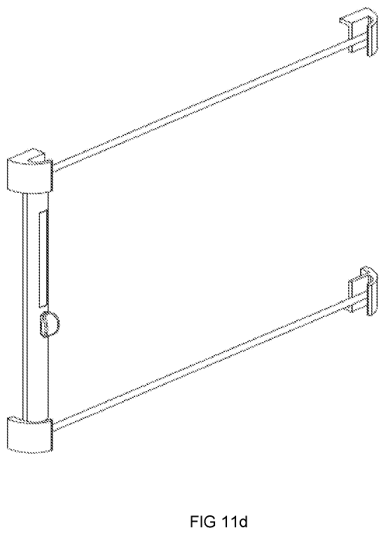

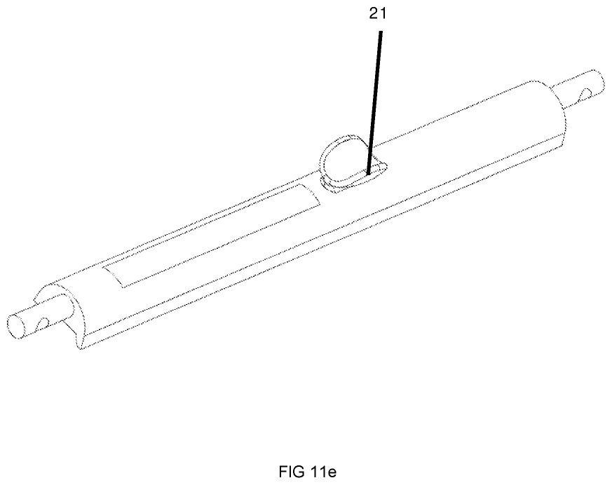

Further embodiments of the present invention, which are designed primarily for use in decontaminating physical objects that are not attached and part of a user are shown in FIGS. 10a to 11e.

FIGS. 10a to 10c show a device 10 of the present invention in which the objects 12 to be decontaminated are shopping trolleys. The device 10 is designed to be installed either outside a grocery or other retail store or centre, most likely proximally located to the store entrance or some other conveniently located position (perhaps in a car park or the like for example), or may also be installed within the store itself. It is already widespread and common practice for shopping trolleys to be stored between uses in similar ‘corrals’, which makes it convenient for shoppers to retrieve a trolley for use, keeps trolleys contained and out of the way when not being used, and allows the shoppers an area to return the trolley after use.

It is envisaged that the device 10 of the present invention would be activated, either by manual switching on or by way of automated trigger, or timer, at which point the trolleys within the decontamination area 16 would be decontaminated by the activation of the UVC lights 18. As is shown in FIG. 10c, a curtain or shield 24 can be used to surround the decontamination area, which in this embodiment is a transparent curtain covering the framework of the device 10, and having an openable end in which the trolleys can be inserted into and retrieved from the decontamination area 16.

Further, another embodiment of the device 10 of the present invention is shown in FIGS. 11a to 11c. In this embodiment, the device 10 is adapted to be installed on a display monitor 12, which would typically be a touch screen display. A decontamination area 16 is formed adjacent to the screen of the display monitor 12 between two arms that extend from respective ends of a body of the device. The arms form a track, along which the body is adapted to move, which as best shown in FIG. 11c allows for the body of the device to be moved therealong and so across the decontamination area 16. In this way, when the UVC light(s) is/are activated and the body is traversed along the tracks of the two arms, the screen of the monitor display within the decontamination area 16 is decontaminated.

Further, an alternate embodiment of the device 10 of the present invention is shown in FIGS. 11d and 11e. In this embodiment, the device 10 is adapted to be installed on a display monitor 12, which would typically be a touch screen display. The device 10 is further adapted to include a finger rest 21 where the user places their forefinger for ease of movement of the body of the device over the surface of the touch screen display. Preference is given to this embodiment as it further reduces the risk of contamination to the user, as the user is more easily able to control or move the body along the tracks by using a single finger (most likely the pointer finger or forefinger), rather than the embodiment shown in FIGS. 11a to 11c, where the user is more likely to grip the tab between forefinger and thumb, in which case both fingers may become contaminated, and it is more difficult to decontaminate the thumb especially, noting that a finger decontamination device may typically be deployed/installed in close proximity to the touch screen decontamination device.

In all foregoing embodiments, the device 10 of the present invention includes a power source, which can be either (or both) of a battery or mains electrical power. The power source can also further includes a solar panel array, which is particularly useful where the device 10 of the present invention will be used remotely or in mobile applications, where there is no mains power, in order to increase the longevity of the device, and to recharge the battery.

However, in accordance with further embodiments of the invention, the object is provided for use in a possibly contaminated environment, and the object is configured to incorporate the power source and the at least one decontamination area. The object further comprises a contact area, which can become contaminated when the object is used within the possibly contaminated environment. The at least one decontamination area is, located within a another portion of the object other than the contact area, and is accessible only when the object has undergone a transformation from one use state to another. The decontamination area includes a void into which the contact area of the object is placed, and further includes the one or more light sources that emit UV-C radiation, which decontaminates the contact area of the object when activated.

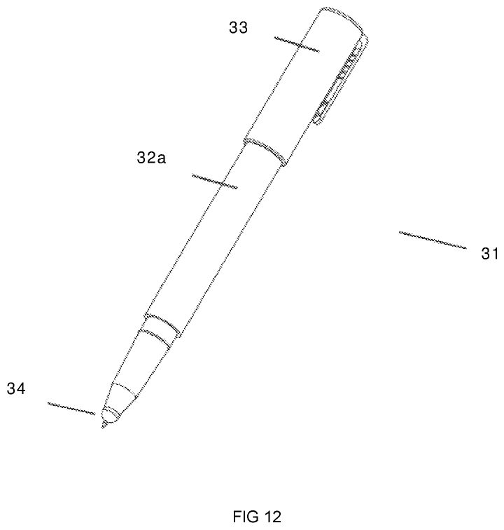

A device 31 in the form of a pen 32a in accordance with an eleventh embodiment of the present invention is shown in FIGS. 12 through 15b. In this eleventh embodiment of the present invention, the device 31 comprises a power source (which is not shown in all of the drawings), and at least one decontamination area 36, which includes a void or chamber 37 into which a contact area 39 of the device 32a can be placed or inserted. The contact area 39 is most vulnerable to becoming contaminated when the device 32a is used within the possibly contaminated environment. In the case of the device 31 being a pen 32a, the contact area 39 comprises the writing tip 34 and a portion of the barrel proximally located to the writing tip. As will be readily appreciated by the skilled person, it is this area of the pen 32a that will typically be gripped by a user when operating the pen to write or draw, and is the surface most likely to become contaminated with viruses or the like, and is similarly the surface most likely to then transmit any pathogens to another user of the pen 32a at a later stage/time.

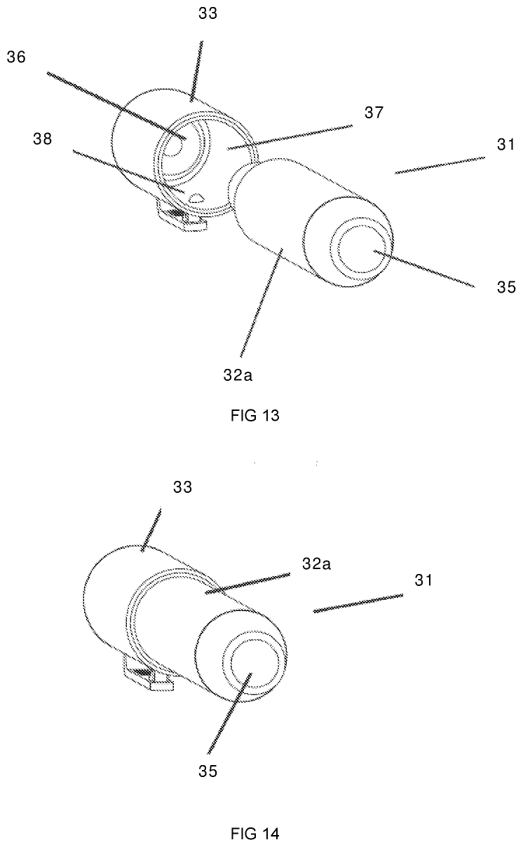

As is best shown in FIGS. 13 to 15B, in this embodiment of the present invention, the decontamination area 36 is located within the pen lid 33. In this way, the decontamination area 36 is accessible only (indeed is only visible) when the pen lid 33 has been removed from the pen barrel 32a. In other words, when the pen lid 33 is installed on the pen barrel 32a, the decontamination area 36 cannot be seen or accessed. In order to access the decontamination area 36, the lid 33 must be removed from the pen barrel 32a, and so it could be said that the device 31 being the pen has undergone a transformation from one use state to another (i.e. pen lid on to pen lid removed).

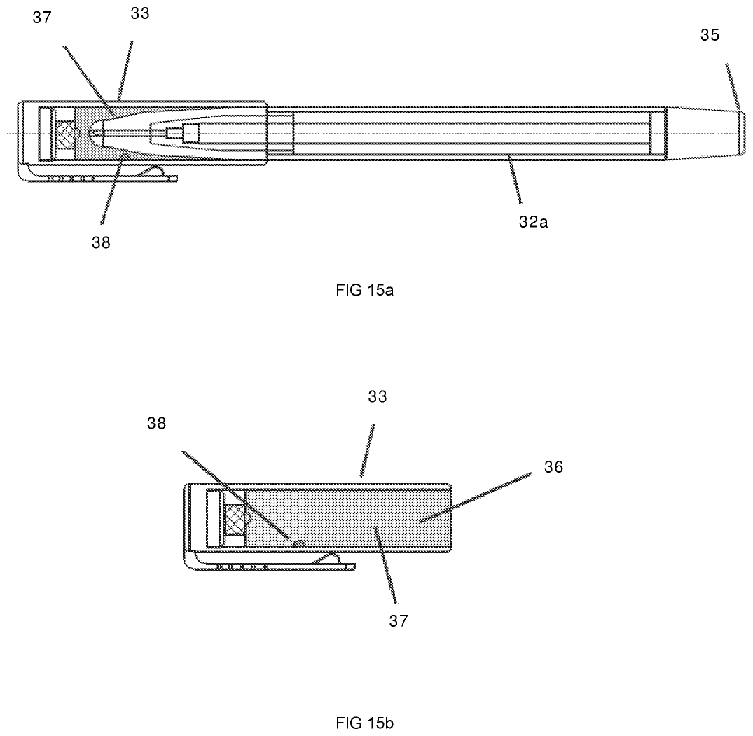

As is best shown in FIGS. 13 and 15a, the decontamination area 36 includes a void 37 into which the contact area of the device is placed, and further includes one or more light sources 38 that emit UV-C radiation, which decontaminates the contact area of the device 31 when activated.

The interaction between the pen barrel 32a and the pen lid 33, within which is located the decontamination area 36 is best shown in FIGS. 13 to 15b.

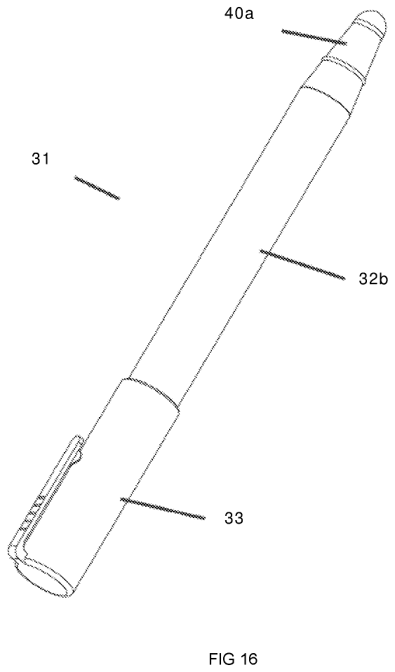

A device 31 in the form of a pen with an incorporated stylus 32b in accordance with a twelfth embodiment of the present invention is shown in FIGS. 16 through 19. In this twelfth embodiment of the present invention, the device 31 comprises a power source (which is not shown in all of the drawings), and at least one decontamination area 36, which includes a void or chamber 37 into which a contact area 39 of the device 32b can be placed or inserted. The contact area 39 is most vulnerable to becoming contaminated when the device 32b is used within the possibly contaminated environment. In the case of the device 31 being a pen with an integrated stylus 32b, the contact area 39 comprises the stylus 40a. As will be readily appreciated by the skilled person, the stylus 40a will typically be one of the surfaces most likely to become contaminated with viruses or the like.

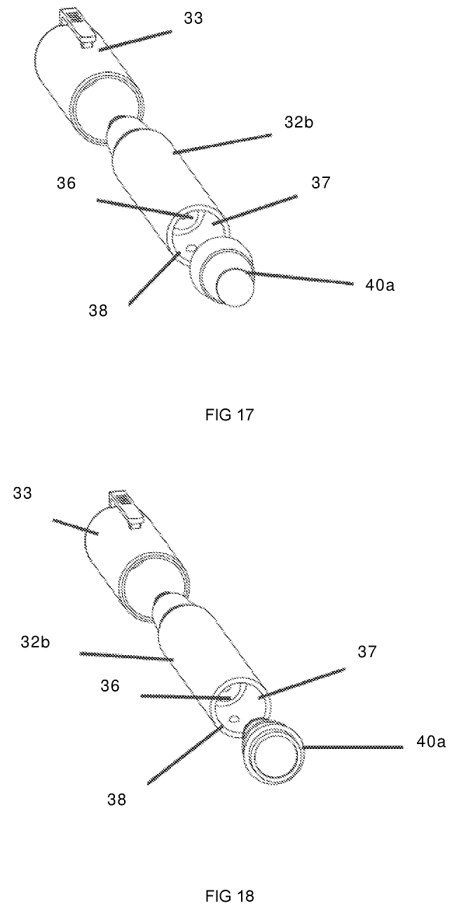

As is best shown in FIGS. 17 to 19, in this embodiment of the present invention, the decontamination area 36 is located within the pen barrel 32b, at position adjacent to the where the stylus 40a is housed when in use. Indeed, the stylus 40a is arranged such that one end of the stylus is adapted to be removably secured within the cavity 37 of the decontamination area 36 when in use, such that the operable end of the stylus is exposed and blocks the entrance to the cavity of the decontamination area. In order to decontaminate the stylus 40a, it is removed from the pen barrel, the stylus is reoriented such that a contact surface thereof is positioned relative to the cavity 37 of the decontamination area 36 such that it can be inserted therein for decontamination.

In this way, the decontamination area 36 is accessible only (indeed is only visible) when the stylus 40a has been removed from the pen barrel 32b. In other words, when the stylus 40a is installed within the pen barrel 32b, the decontamination area 36 cannot be seen or accessed. In order to access the decontamination area 36, the stylus 40a must be removed from the pen barrel 32b, and so it could be said that the device 31 (being the pen with the incorporated stylus) has undergone a transformation from one use state to another (i.e. pen with stylus in operative position, to pen with stylus in decontamination position).

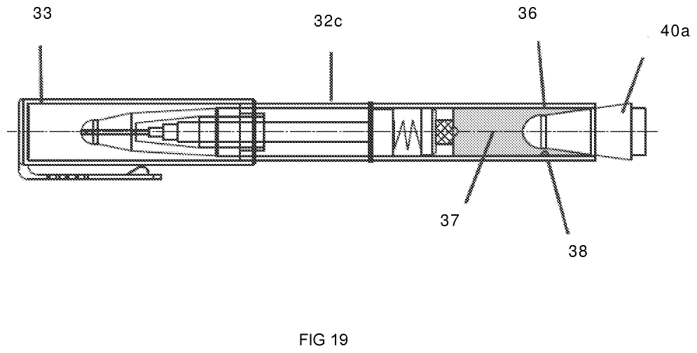

Like with the eleventh embodiment of the invention, as is best shown in FIGS. 17 and 19, the decontamination area 36 includes a void 37 into which the contact area of the device is placed, and further includes one or more light sources 38 that emit UV-C radiation, which decontaminates the contact area of the device 31 when activated.

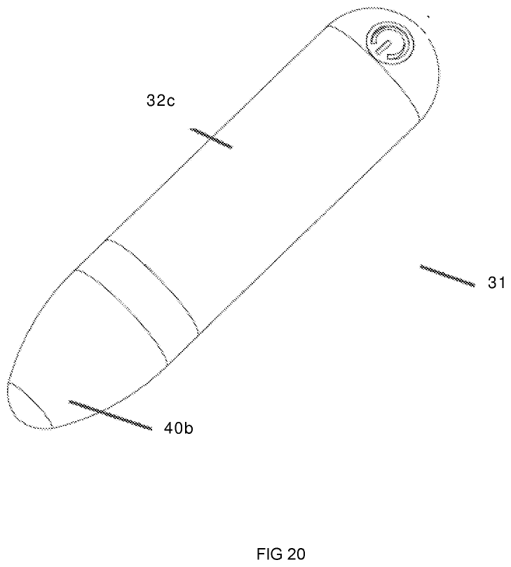

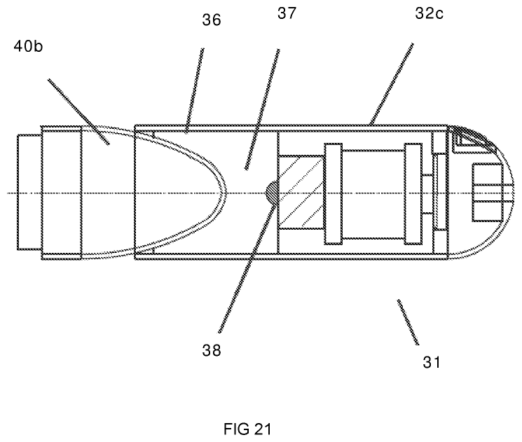

A device 31 in the form of a sexual stimulation device 32c, which includes a body and a removable vibrating tip 40b in accordance with a thirteenth embodiment of the present invention is shown in FIGS. 20 and 21. In this thirteenth embodiment of the present invention, the device 31 comprises a power source (which is not shown in all of the drawings), and at least one decontamination area 36, which includes a void or chamber 37 into which a contact area 39 of the device 32c can be placed or inserted. The contact area 39 is most vulnerable to becoming contaminated when the device 32b is used within the possibly contaminated environment. In the case of the device 31 being a sexual stimulation device 32c, the contact area 39 comprises the removable tip 10b.

Similar to the twelfth embodiment discussed above, the removable vibrating tip 40b is removable from the body of the sexual stimulation device 32c like the stylus 40a, and can be flipped around and inserted into the decontamination area 36 located within the body of the device, at position adjacent to the where the removable tip 40b is housed when in use.

Also similar to the twelfth embodiment discussed above, the removable tip 40b is arranged such that one end of the tip is adapted to be removably secured within the cavity 37 of the decontamination area 36 when in use, such that the operable end of the tip is exposed and blocks the entrance to the cavity of the decontamination area. In order to decontaminate the tip 40b, it is removed from the body of the sexual stimulation device 32c, it is reoriented such that a contact surface thereof is positioned relative to the cavity 37 of the decontamination area 36 such that it can be inserted therein for decontamination.

In this way, the decontamination area 36 is accessible only (indeed is only visible) when the removable tip 40b has been removed from the body of the sexual stimulation device 32c. In other words, when the tip 40b is installed within the body, the decontamination area 36 cannot be seen or accessed. In order to access the decontamination area 36, the tip 40b must be removed from the body of the sexual stimulation device 32c, and so it could be said that the device 31 (being the sexual stimulation device with a removable tip) has undergone a transformation from one use state to another.

Like with the eleventh and twelfth embodiments of the invention, as is best shown in FIGS. 20 and 21, the decontamination area 36 includes a void 37 into which the contact area of the device is placed, and further includes one or more light sources 38 that emit UV-C radiation, which decontaminates the contact area of the device 31 when activated.

The use of UV-C radiation to inactivate microorganisms so they are no longer capable or replicating and causing adverse health effects is well known. UV-C radiation has been used for decades to disinfect surfaces and sanitise water and air for example. Ultra-Violet light is invisible to the human eye and is divided into UV-A, UV-B and UV-C. UV-C radiation is found within 100-280 nm range, and its germicidal action is maximized at 254 nm with reductions on either side of this wavelength.

Whilst it is not shown in the drawings, the decontamination area 36 can further include an automated sensor to detect when the object is introduced therein. When the object is detected, the sensor will automatically activate the one or more the light sources 38.

To provide a greater sense of user satisfaction, the device 40 further includes a second light source (that is not shown in the drawings) that emits visible light when activated. This light source is designed to illuminate at the same time the UV-C radiation is being emitted by the UV-C light source, and thus the visible light indicates to a user when the UV-C light source is activated. This light is preferably bright violet or blue, and most preferably is the visible spectrum UV-A or UV-B light, which user's associate with sterilizing and decontamination.

This second visible light source is a light source located somewhere on the body of the device, which acts in a similar way to a power light indicator.

The device 31 can further comprise a digital display (that is not shown in the drawings) that conveys information about the duration of the period of activation of the UV-C light source to the user in real time. The digital display may, for example, includes a countdown timer showing the duration of the period of activation of the at least one UV-C light source 38.

In each of embodiments eleven through thirteen, the device 31 of the present invention includes a power source, which is in the form of a battery, which can be either a onetime use battery or a rechargeable battery.

While the invention has been described with reference to preferred embodiments above, it will be appreciated by those skilled in the art that it is not limited to those embodiments, but may be embodied in many other forms, variations and modifications other than those specifically described. The invention includes all such variation and modifications. The invention also includes all of the steps, features, components and/or devices referred to or indicated in the specification, individually or collectively and any and all combinations or any two or more of the steps or features.

In this specification, unless the context clearly indicates otherwise, the word “comprising” is not intended to have the exclusive meaning of the word such as “consisting only of”, but rather has the non-exclusive meaning, in the sense of “including at least”. The same applies, with corresponding grammatical changes, to other forms of the word such as “comprise”, etc.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Any promises made in the present document should be understood to relate to some embodiments of the invention, and are not intended to be promises made about the invention in all embodiments. Where there are promises that are deemed to apply to all embodiments of the invention, the applicant/patentee reserves the right to later delete them from the description and they do not rely on these promises for the acceptance or subsequent grant of a patent in any country.

1.-31. (canceled)

32. A system for decontaminating an object, the system comprising:
a power source, and
at least one decontamination area; and
characterized in that the at least one decontamination area includes one or more light sources that emit UV-C radiation.

33. The system according to claim 32, wherein the system includes a device for decontaminating a surface of the object, said device incorporating the power source and the at least one decontamination area, and
said device decontaminating the surface of the object when the object is located in the decontamination area and the one or more light sources is activated.

34. The system according to claim 33 wherein the decontamination area includes a void into which the surface of the object is placed.

35. The system according to claim 34 wherein the decontamination area further includes an automated sensor to detect when the object is introduced therein, and when the object is detected, automatically activating the one or more light sources.

36. The system according to claim 33 wherein the device further including a second light source that emits visible light to indicate to a user when the at least one UV-C light source is activated.

37. The system according to claim 36 wherein the second visible light source is located within the decontamination area such that the decontamination area, and the object positioned therein, is illuminated by the visible light simultaneously with the UV-C radiation emitted from the UV-C light source.

38. The system according to claim 33 wherein the device further comprising a digital display that conveys information about the duration of the period of activation of the UV-C light source to the user in real time, which includes a countdown timer showing the duration of the period of activation of the at least one UV-C light source.

39. The system according to claim 33 wherein the object is one or more of a users' hand or finger, an EFTPOS terminal, a payment card, or a mobile or cellular telephone.

40. The system according to claim 33 wherein the power source is selected from one or more of a battery or mains electrical power.

41. The system according to claim 33 wherein the decontamination area includes a shield at least partially obstructing visual access into the decontamination area in order to protect the user's eyes from the UV-C, which can be damaging if looked at directly.

42. The system according to claim 33 wherein the device further comprising a movement sensor that detects when the device has been moved or detects a movement adjacent to the device, and deactivates the UV-C light source.

43. The system according to claim 33 wherein the device further comprising a body within which the one or more light sources are located, and having first and second rail members laterally extending from opposite ends thereof, the rail members defining the perimeter of the decontamination area, such that the device is adapted to be installed on the peripheral edges of a display monitor, and wherein the body is adapted to extend along the first and second rails such that, when activated, the light source passes across a display screen surface of the monitor display, decontaminating the same.

44. The system according to claim 32 wherein the object is provided for use in a possibly contaminated environment, and the object is configured to incorporate the power source and the at least one decontamination area,
wherein the object further comprises a contact area, which can become contaminated when the object is used within the possibly contaminated environment;
and wherein the at least one decontamination area is located within a portion of the object other than the contact area, and is accessible only when the object has undergone a transformation from one use state to another, and the decontamination area includes a void into which the contact area of the object is placed, and further includes the one or more light sources which decontaminate the contact area of the object when activated.

45. The system according to claim 44 wherein the contact area can be removed from the object, and once removed, reoriented such that a contact surface thereof is positioned relative to the void of the decontamination chamber such that it can be inserted therein for decontamination

46. The system according to claim 44 wherein the object is a pen, the contact area comprises the writing tip and a portion of the barrel proximally located to the writing tip, and the decontamination area is located within the pen lid, wherein a first use state is where the lid is engaged with the end of the pen distally located from the writing tip, and the second use state is where the lid is engaged with the end of the pen adjacent the writing tip.

47. The system according to claim 44 wherein the object is a pen which includes an integrated stylus positioned at the end of the pen distally located from the writing tip, the contact area comprises the stylus, and the decontamination area is located within the barrel of the pen adjacent to the stylus, wherein a first use state is where the stylus is engaged with the end of the pen distally located from the writing tip, and the second use state is where the stylus has been removed from the end of the pen and reoriented such that a contact surface thereof is positioned relative to the void of the decontamination chamber such that it can be inserted therein for decontamination.

48. The system according to claim 44 wherein the object is a sexual stimulation device comprising a body and a removable vibrating tip, the contact area is the removable vibrating tip which can be inserted into one or more body cavities during use, and the decontamination area is located within the body of the sexual stimulation device adjacent to the removable vibrating tip, wherein a first use state is where the removable vibrating tip is engaged with the end of the body of the sexual stimulation device, and the second use state is where the removable vibrating tip has been removed from the end of the body and reoriented such that a contact surface thereof is positioned relative to the void of the decontamination chamber such that it can be inserted therein for decontamination.

49. The system according to claim 44 wherein the decontamination area further includes an automated sensor to detect when the object is introduced therein, and when the object is detected, automatically activating the one or more light sources.

50. The system according to claim 44 wherein the object further including a second light source that emits visible light to indicate to a user when the at least one UV-C light source is activated.

51. The system according to claim 44 wherein the device further comprising a digital display that conveys information about the duration of the period of activation of the UV-C light source to the user in real time, which includes a countdown timer showing the duration of the period of activation of the at least one UV-C light source.
